(19) United States
(12) Patent Application Publication
(10) Pub. No.: US 2021/0257081 A1
(43) Pub. Date: Aug. 19, 2021

(54) APPLIED BEHAVIOR ANALYSIS (ABA) MEDICAL NECESSITY ASSESSMENT AND DOSAGE CALCULATOR

(71) Applicant: Rethink Autism, Inc., New York, NY (US)

(72) Inventors: Daniel A. Etra, New York, NY (US); Eran Rosenthal, New York, NY (US); Jamie C. Pagliaro, New York, NY (US)

(21) Appl. No.: 17/174,962

(22) Filed: Feb. 12, 2021

Related U.S. Application Data
(60) Provisional application No. 62/976,675, filed on Feb. 14, 2020.

(51) Int. Cl. G16H 20/70; G16H 10/60; G16H 10/20; G16H 20/30; G16H 50/30

(52) U.S. Cl. CPC G16H 20/70; G16H 10/60; G16H 50/30; G16H 20/30; G16H 10/20

(57) ABSTRACT

1. A computer-implemented behavioral health assessment for ABA therapy method comprising: receiving a set of responses from a queried condition of an individual to generate an ABA score established by an human-generated examination of the individual; prescribing a ABA treatment based on the ABA score; correlating, via a processor, the ABA treatment to a corpus of ABA treatment data; and calibrating the recommended ABA treatment based on a set of computer-implemented feedback associated with the ABA score associated with the individual, via a processor, providing at least a ABA treatment recommendation for modification of the ABA treatment.

### CROSS REFERENCE TO RELATED APPLICATIONS

This application claims the benefit of U.S. Provisional Application No. 62/976,675, Filed Feb. 14, 2020, and the entire contents of which are incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and, in the drawings, that form a part of this document: Copyright 2020, Rethink Autism Inc., All Rights Reserved

### TECHNICAL FIELD

This document pertains generally, but not by way of limitation, to optimizing assessment and treatment of individuals requiring counseling and specific treatment regimens when suffering from autism or other spectrum related disorders

### BACKGROUND

### Autism Spectrum Disorders

Autism is a complex neurological and developmental disorder with behavior phenotypes including impairments in social skills, communication difficulties, and repetitive, rigid, restricted behaviors. Across the nation, autism occurs in an estimated one in 68 children; boys are nearly five times more likely to be diagnosed with autism than girls, and autism can affect any ethnicity. Autism is identified as a deficit in communication that ranged from a delay to total lack of verbal communication, restricted interests, and inconsistencies in responding. (Other impairments include atypical development in responding, vocalization, play, initiation, and response to name.)

Autism prognosis is dependent on the early identification of the disorder, thus supporting the continued need to begin evidence-based treatment as early as possible. Early intervention is a critical time in development in which the modifiability of developmental trajectory is high, thus allowing the learners the opportunity to be placed on a normative developmental trajectory. As the gap in development increases between a child's age and a child's development, remediation of concerns become more challenging.

### ABA Definitions and Treatment Models

ABA is an acronym for Applied Behavioral Analysis, and it is often described as the “gold standard” for autism treatment. Applied Behavioral Analysis (ABA) is a system of autism treatment based on behaviorist theories which, simply put, state that desired behaviors can be taught through a system of rewards and consequences. ABA can be thought of as applying behavioral principles to behavioral goals and carefully measuring the results.

While the idea of using rewards and consequences to teach behavior is probably as old as human civilization, the idea of carefully applying rewards and consequences to achieve specific, measurable goals is relatively new.

### How ABA Works

Discrete trials ABA is still in use in some settings, and for some children. Other forms of ABA, however, are becoming increasingly popular. In addition, rather than providing 1:1 therapy in a classroom or office, many therapists are now administering ABA in natural settings such as playgrounds, cafeterias, and community locations. This approach makes it easier for children to immediately use the skills they learn in a real-world situation.

There are two commonly accepted models of ABA treatment; Comprehensive and Focused. Both models have variations in program dimensions such as peer involvement, parent involvement or delivery approach (i.e. structured vs naturalistic.) Comprehensive treatment is generally delivered in large dosages ranging from 25-40 hours per week and addresses a large number of domains including communication, social, behavioral and academic. The length of treatment varies, but at minimum treatment should occur for two years.

Comprehensive ABA treatment targets skill acquisition across multiple domains by increasing communication, social, emotional, adaptive and cognitive functioning skills as well as reducing maladaptive behaviors that impact learning, client health and safety, family and community safety and functional independence. The cost of this program can exceed $50,000 per year, but it has also been suggested that future healthcare cost can be greater over longer periods of time if the treatment is not provided.

A second treatment model is Focused ABA Treatment. Different from Comprehensive ABA, Focused treatment addresses a limited number of behavioral targets. It is optimal for individuals who need only to acquire a limited number of pivotal skills or for those who have an acute problem behavior that affects the health and safety of themselves or others. Dosage of treatment will vary, but on average occurs at 10-25 hours per week.

A problem identified in this treatment regime is that no set of normalized assessments and treatment schedules have been normalized between the payer (insurance) community and the provider (treatment) community. There are also large discrepancies between the services provided between various providers in the community as well as the levels of services authorized by the payer communities; this can be quantified as a larger problem.

### BRIEF SUMMARY

The inventors of this solution have recognized the advantages of providing a standardized set of assessments for both Focused ABA and Comprehensive ABA filings. The inventors have also recognized the advantages of providing a solution that allows a normalization of treatment both for providers and payers to allow for easy approval and easy delivery of commonly prescribed services. The inventors have also recognized the advantages of providing the assessments and the approval of treatments requested via a mobile device to optimize the ready gathering, dissemination and decision making by providers and the approval of prescribed treatment. In addition, the inventors have also recognized that using a corpus of recommendations and treatment data, and training the solution, using a computer system, to optimize its recommendations and receiving the feedback allows the solution to make better and better recommendations.

### BRIEF DESCRIPTION OF THE DRAWINGS

To easily identify the discussion of any particular element or act, the most significant digit or digits in a reference number refer to the figure number in which that element is first introduced.

FIG. 1 illustrates an Assessment Flow 100 in accordance with one example of the solution.

FIG. 2 illustrates a Networked Solution 200 in accordance with one example of the solution.

FIG. 3 illustrates a Solution processing environment 300 in accordance with one example of the solution.

FIG. 4 illustrates a Solution block diagram 400 in accordance with one example of the solution.

FIG. 5 illustrates a Solution machine 500 in accordance with one example of the solution.

FIG. 6 illustrates a Solution machine-learning program 600 in accordance with one example of the solution.

FIG. 7 illustrates an Introduction Screen User Interface 700 in accordance with one example of the solution.

FIG. 8 illustrates an Open Assessment User Interface 800 in accordance with one example of the solution.

FIG. 9 illustrates an Assessment Choice User Interface 900 in accordance with one example of the solution.

FIG. 10 illustrates a Treatment Model Input User Interface 1000 in accordance with one example of the solution.

FIG. 11 illustrates a Planning Data User Interface 1100 in accordance with one example of the solution.

FIG. 12 illustrates a Current Status User Interface 1200 in accordance with one example of the solution.

FIG. 13 illustrates an Environmental Considerations User Interface 1300 in accordance with one example of the solution.

FIG. 14 illustrates a Clinical Data User Interface 1400 in accordance with one example of the solution.

FIG. 15 illustrates a Result and Recommendation User Interface 1500 in accordance with one example of the solution.

FIG. 16 illustrates a Results Summary User Interface 1600 in accordance with one example of the solution.

FIG. 17 illustrates a Comprehensive Report User Interface 1700 in accordance with one example of the solution.

FIG. 18 illustrates a charting interface example 1800 in accordance with one example of the solution.

### DETAILED DESCRIPTION

Embodiments of this solution may be implemented in one or a combination of hardware, firmware and software. Embodiments may also be implemented as instructions stored on a computer-readable storage device, which may be read and executed by at least one processor to perform the operations described herein. A computer-readable storage device may include any non-storing information in a form readable by a machine (e.g., a computer). For example, a computer-readable storage device may include read-only memory (ROM), random-access memory (RAM), magnetic disk storage media, optical storage media, flash-memory devices, cloud servers or other storage devices and media. Some embodiments may include one or more processors and may be configured with instructions stored on a computer-readable storage device. The following description and the referenced drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The present solution automates and improves the efficacy, the predictability of treatment and efficiency of insurance claims around autism.

### Autism Spectrum Disorders

Autism is a complex neurological and developmental disorder with behavior phenotypes including impairments in social skills, communication difficulties, and repetitive, rigid, restricted behaviors. Across the nation, autism occurs in an estimated one in 68 children; boys are nearly five times more likely to be diagnosed with autism than girls, and autism can affect any ethnicity. Autism is identified as a deficit in communication that ranged from a delay to total lack of verbal communication, restricted interests, and inconsistencies in responding. Other impairments include atypical development in responding, vocalization, play, initiation, and response to name.

Autism prognosis is dependent on the early identification of the disorder, thus supporting the continued need to begin evidence-based treatment as early as possible. Early intervention is a critical time in development in which the modifiability of developmental trajectory is high, thus allowing the learners the opportunity to be placed on a normative developmental trajectory. As the gap in development increases between a child's age and a child's development, remediation of concerns become more challenging. By identifying developmental delays early in the child's life, one may decrease future risk of other medical and behavioral conditions, and therefore this surveillance should be routine and include specific inquiries related to the core symptoms of autism.

Autism is a chronic disorder in which on-going medical and social management of symptoms may be lifelong and is a necessary aspect of care. Children diagnosed with autism have shown improved health and developmental outcomes through the use of early, intensive and direct evidence-based treatment.

### Comorbidity

Because of the complexity of autism, comorbid conditions are common; however, these comorbidities may be difficult to detect given the social and communication impairments specific to autism. Autism is often accompanied with a range of coexisting behavioral difficulties including aggression, self-injury, hyperactivity, and obsessive-compulsive disorders. Individuals with autism may have frequent co-occurring medical conditions that increase mortality rates amongst this population. For example, seizures are prevalent in nearly 20-25% of the autism population. Other conditions include anxiety, disrupted sleep, immune dysregulation, allergies, gastrointestinal problems, metabolic abnormalities, and dysautonomia. Due to the extreme heterogeneity present in autism spectrum disorders, treatment of comorbid conditions varies, and may be complex and continuous, requiring ongoing collaboration and care with a primary care physician.

With the prevalence occurring in 50-80% of patients with autism, disruptive sleep remains one of the most significant and problematic health concerns amongst parents and professionals treating autism. Additionally, sleep disturbances in children are a key symptom that assists in the identification of autism in young children under the age of two. Sleep difficulty is not specifically restricted to just early childhood but may also be a lifelong concern for families and as such may result in increased parental stress. In young children with autism, families report sleep behavior concerns specifically related to sleep anxiety, bedtime resistance, and night awakenings.

The present solution automates and improves the efficacy of the ABA treatments prescribed, improves the predictability of treatment by providing statistical output and guidelines based on various data and studies and improves efficiency and predictability of authorization requests and payments of insurance claims.

### General Description of the ABA Treatment Regime

ABA is considered an evidence-based best practice treatment by the US Surgeon General and by the American Psychological Association. “Evidence based” means that ABA has passed scientific tests of its usefulness, quality, and effectiveness. ABA therapy includes many different techniques. All of these techniques focus on antecedents (what happens before a behavior occurs) and on consequences (what happens after the behavior). More than 20 studies have established that intensive and long-term therapy using ABA principles improves outcomes for many but not all children with autism. “Intensive” and “long term” refer to programs that provide 25 to 40 hours a week of therapy for 1 to 3 years. These studies show gains in intellectual functioning, language development, daily living skills and social functioning. Studies with adults using ABA principles, though fewer in number, show similar benefits.

There are two commonly accepted models of ABA treatment; Comprehensive and Focused. Both models have variations in program dimensions such as peer involvement, parent involvement or delivery approach (i.e. structured vs naturalistic) Comprehensive treatment is generally delivered in large dosages ranging from 25-40 hours per week and addresses a large number of domains including communication, social, behavioral and academic. The length of treatment varies, but at minimum treatment should occur for two years. Comprehensive ABA treatment targets skill acquisition across multiple domains by increasing communication, social, emotional, adaptive and cognitive functioning skills as well as reducing maladaptive behaviors that impact learning, client health and safety, family and community safety and functional independence. The cost of this program can exceed $50,000 per year, but it also has been suggested that future healthcare cost can be greater over longer periods of time if the treatment is not provided.

A second treatment model is Focused ABA Treatment. Different from Comprehensive ABA, Focused treatment addresses a limited number of behavioral targets. It is optimal for individuals who need only to acquire a limited number of pivotal skills or for those who have an acute problem behavior that affects the health and safety of themselves or others. Dosage of treatment will vary, but on average occurs at 10-25 hours per week.

### Medical Necessity Assessment Criteria and its Variances

The various insurers and state agencies publish varied requirements to determine medical necessity. For example, here below is a set of AETNA criteria that would be used to validate ABA therapy claims and be used by the solution to validate medical necessity.
1. There must be a diagnosis of a condition on the Autism Spectrum (ICD-9: 299 through 299.9; ICD-10: F84 through F84.9)
2. There are identifiable target behaviors having an impact on development, communication, interaction with typically developing peers or others in the child's environment, or adjustment to the settings in which the child functions, such that the child cannot adequately participate in developmentally appropriate essential community activities such as school. The ABA is not custodial in nature (which AETNA defines as care provided when the member “has reached the maximum level of physical or mental function and such person is not likely to make further significant improvement” or “any type of care where the primary purpose of the type of care provided is to attend to the member's daily living activities which do not entail or require the continuing attention of trained medical or paramedical personnel.”) Plan documents may have variations on this definition and need to be reviewed.
3. Parent(s) (or guardians) must be involved in training in behavioral techniques so that they can provide additional hours of intervention.
4. There is a time limited, individualized treatment plan developed that is child-centered, strengths-specific, family-focused, community-based, multi-system, culturally-competent, and least intrusive; where specific target behaviors are clearly defined; frequency, rate, symptom intensity or duration, or other objective measures of baseline levels are recorded, and quantifiable criteria for progress are established; describing behavioral intervention techniques appropriate to the target behavior, reinforcers selected, and strategies for generalization of learned skills are specified; and there is documentation of planning for transition through the continuum of interventions, services, and settings, as well as discharge criteria.
5. There is involvement of community resources to include at a minimum, the school district if the child is 3 or older, or early intervention if not.
6. Services must be provided directly or billed by individuals licensed by the state or certified by the Behavior Analyst Certifying Board unless state mandates, plan documents or contracts require otherwise. If state mandates, plan documents or contracts allow authorization for services that are not directly provided by individuals licensed by the state or certified by the Behavior Analyst Certifying Board, there must be supervision of the unlicensed or non-certified providers, unless state mandates, plan documents or contracts require otherwise. Supervision is to be documented and is defined as at least one hour of face-to-face supervision of the unlicensed or non-certified provider by a certified behavior analyst or licensed psychologist for each ten hours of behavioral therapy by the supervised provider, and at least one hour a month face-to-face, on-site with the patient.

As may be appreciated, criteria vary between insurance carriers and the various states, and thus the solution must ingest the criteria of many different providers and states to reliably help a clinician (provider) prescribe ABA therapy in the way best suited to the patient and to the payer (insurer.)

### Treatment/Assessment Flow

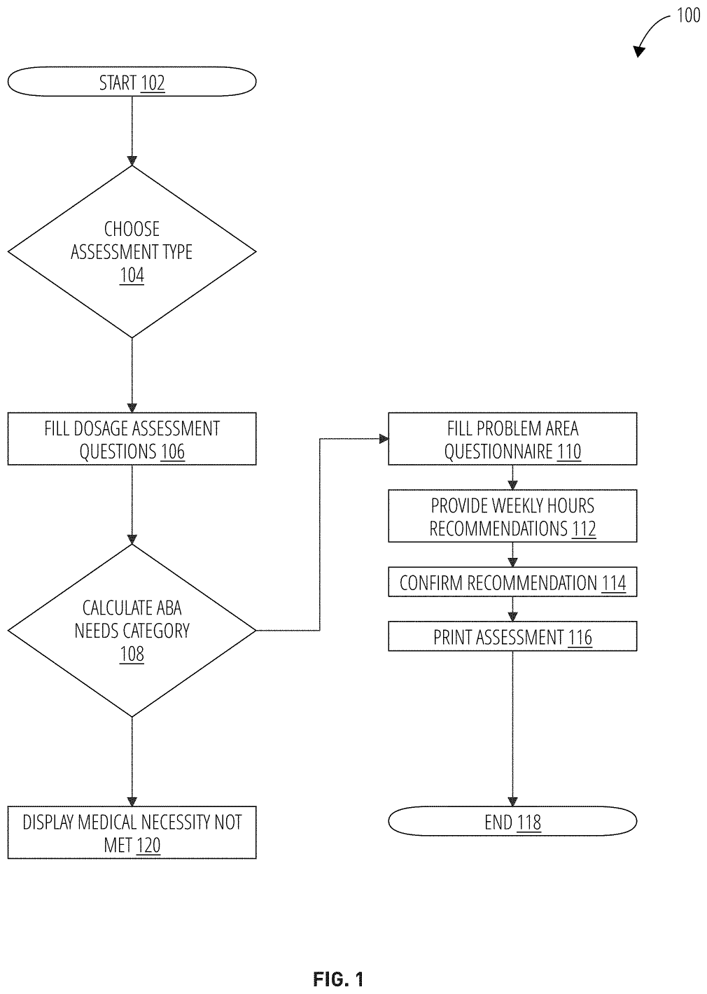

FIG. 1 shows The Assessment Flow 100 comprises a Start Step 102, a Choose Assessment 104, a Dosage Assessment Step 106, a Calculation of Needs 108, an Uncover Problem Area Step 110, a Recommendation Step 112, a Confirmation step 114, an Assessment Output 116, an End 118, and a Medical Necessity Not Met Step 120. In this Assessment Flow 100, the user Choose Assessment 104 step must select a set of service options as the recommended dosage may differ based on whether the user is initially starting the treatment program or re-authorizing for new set of hours

Options comprise an Initial Service Authorization or a Service Re-authorization. The Dosage Assessment Step 106 step uses a Questionnaire that has different sets of questions for each assessment type based on the impact on the needed dosage, and every question has a different weight. A weighted score for each question is based on the weight and the answer scoring. Once the Dosage Assessment Step 106 is complete, the process moves to an ABA Calculation of Needs 108 step where scoring of the question answers is performed resulting in three outcomes. A score in excess of 7 takes the solution to a subsequent comprehensive ABA step where the Uncover Problem Area Step 110 requires a subsequent questionnaires to fully define a set of comprehensive problems and treatments. A score between 4-7 results in a focused ABA where the Uncover Problem Area Step 110 requires a subsequent questionnaire to fully uncover a narrower set of focused problems and treatments for the subject of the Assessment Flow 100. A score less than 4 will result in a Medical Necessity Not Met Step 120 being completed. The Uncover Problem Area Step 110 comprises asking a user to answer questions related to the severity of each problem are, which along with the Dosage Category will determine the number of hours needed for each area. The Assessment Flow 100 also utilizes a machine learning step at the end as to whether a provider or payer agrees with the recommendation and the Assessment Output 116

Additional optional steps comprise a) Fine-tune hours recommendations for the different skills/behaviors categories b) receive recommendations and alerts when the solution algorithms identify an outlier, c) identify and isolate non-clinical factors influencing clinician recommendations, d) recommendations for improving insurance coverage, and e) monitor overall clinician agreement with system recommendations as an indicator of process performance and source of feedback for adjustments to process and questionnaire.

A non-limiting example of the types of data metrics is incorporated in the solution is listed by primary groups of cohorts and other segmentation fields. The solution also allows filtration of results via this set of data fields.

Patient Data Fields Collected and Coordinated.
Age Cohorts
Age at start of treatments
Current age
Utilization Cohorts
Utilized Hours/Authorized Hours
Utilized Hours/Recommended Hours
Severity Cohorts
Treatment Cohorts
Comprehensive vs. Focused ABA
MNA Recommended Hours
Other assessments
By Curriculum Area
By Gender
By Payer
By Individual Payer/Payer Group
Commercial vs. Public vs. Private Pay

Another view of the solution's data fields that are gathered on an exemplar patient. The patient is recorded by observation by the professional as well as by the parents of the patient. In the table below, a sample of the types of standard therapy measurements are listed

As may be readily intuited, the solution can expanded or contracted based on the sophistication or cohort that the individual is assigned to. The tables below represent examples of other data items to be captured for the ABA ecosystem. This set of data items includes the infrastructure data to filter by patient, by clinician (provider), by state, by payer, by practice and by ABA benchmarks.

The data fields below capture various aspects of the ABA therapy, comprising metrics of acquisition or improvement of new skills by the patient are captured as well as capturing the behavioral reduction metrics of the individual and the trends related to improving various behaviors distinct from the skills metrics. Both sets of metrics are generally used by both providers and payers to create and analyze unique sets of targets, objectives and plans.

| | | | |
|---|---|---|---|
| By Child | By Provider | By Practice | Benchmarks |

RATE OF SKILLS ACQUISITION
1. Skills Targets Mastered/Time
a) Elapsed time since start of treatment
b) Hours of therapy (billable hours)
2. Skills Objectives Mastered/Time
a) Elapsed time since start of treatment
b) Hours of therapy (billable hours)
3. Skills Goals Mastered/Time
a) Elapsed time since start of treatment
b) Hours of therapy (billable hours)

Clinical fields are also captured as well as performance metrics of the patient to assist in justifying the appropriateness of therapy and to record the patient's engagement and successes using to measure improvement.

RATE OF BEHAVIOR REDUCTION
1. Behavior Objectives Mastered/Time
a) Elapsed time since start of treatment
b) Hours of therapy (billable hours)
2. Behavior Goals Mastered/Time
a) Elapsed time since start of treatment
b) Hours of therapy (billable hours)

Insurer fields are also captured to track utilization of the approved/used hours under MNA assessment.

UTILIZATION
1. Hours Used/Hours Authorized
2. Hours Used/Hours Recommended by MNA

Analytic and variance data fields are also provided as feedback to the insuring entity as well as benchmark and other outcomes for the purposes of tracking patient and therapy process and progress.

VARIANCE FROM MNA RECOMMENDATION
1. (Hours Requested − Hours Recommended)/Hours Recommended
2. (Absolute Value of (Hours Requested −
Hours Recommended))/Hours Recommended
Outputs
Calculated Statistics (and vs. Benchmarks)
Time Series and Trend lines (and vs. Benchmarks)
Scatter Plots

### Dosage Calculator Operating Environments

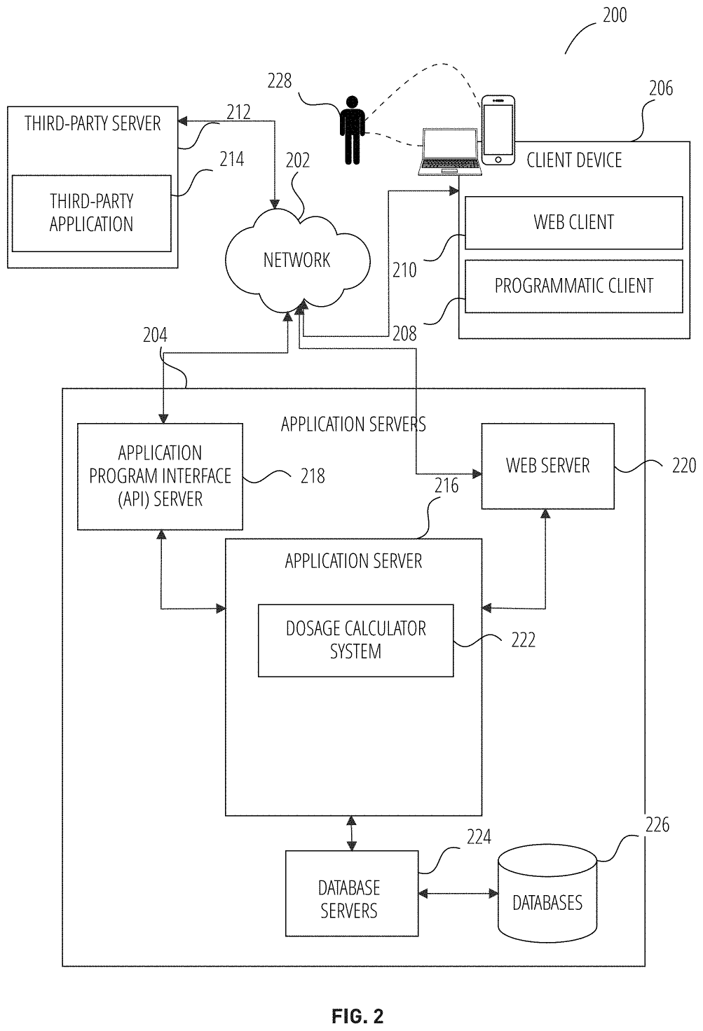

FIG. 2 is a diagrammatic representation of a networked computing environment 200 in which some examples of the present solution may be implemented or deployed.

One or more application servers 204 provide server-side functionality via a network 202 to a networked user device, in the form of a client device 206 that is accessed by a user 228. A web client 210 (e.g., a browser) and a programmatic client 208 (e.g., an “app”) are hosted and execute on the web client 110.

An Application Program Interface (API) server 218 and a web server 220 provide respective programmatic and web interfaces to application servers 204. A specific application server 216 hosts a Dosage Calculator system 222, which includes components, modules and/or applications.

The web client 210 communicates with the Dosage Calculator system 222 via the web interface supported by the web server 220. Similarly, the programmatic client 208 communicates with the Dosage Calculator system 222 via the programmatic interface provided by the Application Program Interface (API) server 218. The third-party application 214 may, for example, be a Provider or Payer third party system accepting treatment regimens or an insurance claim relating to a patient.

The application server 216 is shown to be communicatively coupled to database servers 224 that facilitates access to an information storage repository or databases 226. In an example embodiment, the databases 226 includes storage devices that store information to be published and/or processed by the Dosage Calculator system 222.

Additionally, a third-party application 214 executing on a third-party server 212, is shown as having programmatic access to the application server 216 via the programmatic interface provided by the Application Program Interface (API) server 218. For example, the third-party application 214, using information retrieved from the application server 216, may support one or more features or functions on a website hosted by the third party.

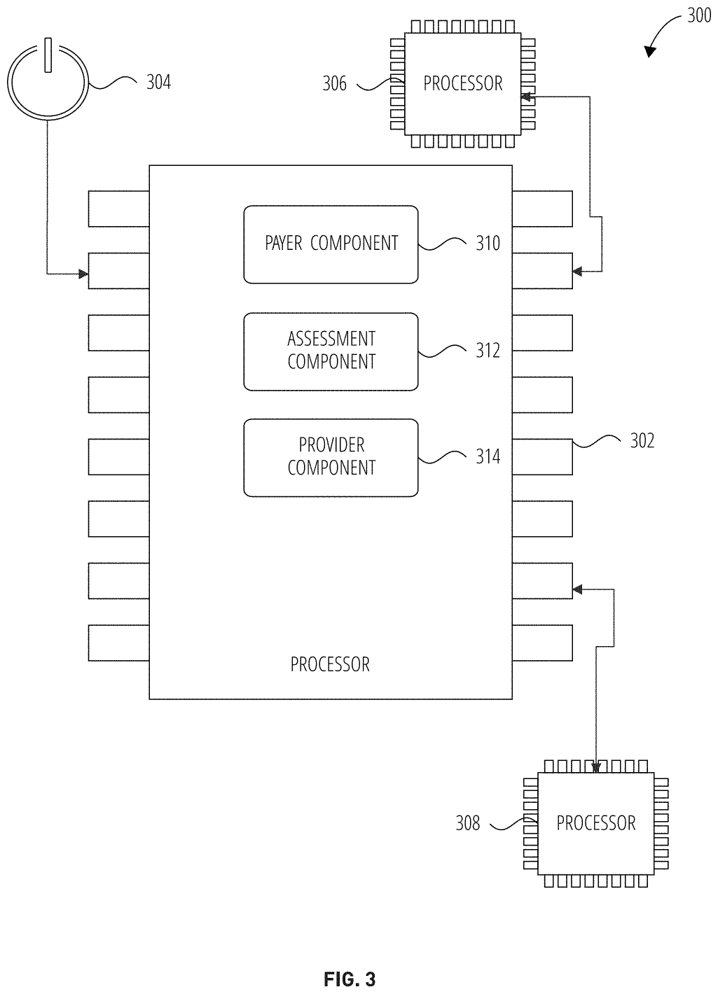

Turning now to FIG. 3, a diagrammatic representation of a multiprocessor processing environment 300 of the present solution is shown, which includes the Processor 306, the Processor 308, and a Processor 302 (e.g., a GPU, CPU or combination thereof).

The Processor 302 is shown to be coupled to a power source 304, and to include (either permanently configured or temporarily instantiated) modules, namely a Payer execution component 310, a Assessment component 312, and a Provider 408

component 314. The Assessment execution component operationally generates Assessment Flow 100 and manages the user, the Payer execution component 312 operationally generates Insurance claims and approvals, and the Provider execution component 314 operationally generates Clinical dosages and manages Provider Workflow. As illustrated, the Processor 302 is communicatively coupled to both the Processor 306 and Processor 308, and receives commands from the Processor 306, as well as commands from the Processor 308.

The following example is a non-limiting manner of enabling this solution. The solution also contemplates using other analogous hardware or software implementation than those explicitly mentioned.

### TECHNOLOGY AND INFRASTRUCTURE

This example solution is delivered over the Internet using virtual machines (VMs) for web and database servers, (e.g., AZURE web apps to host web applications, and AZURE blob storage for content storage. The solution deploys dynamically generated application features as well as derived or published content via various servers. The web applications are developed with any modern app development environment (e.g., Customer-facing solutions are developed in .NET Core, MVC Framework, C #, Angular, and SQL formats using Web 2.0 functionality standards.

### Data Centers

An example of the data center implementation uses 3^{rd} party data centers like those provided by GOOGLE, AMAZON, MICROSOFT vendors that comply with stringent data privacy requirement from the jurisdiction served by the platform.

### Servers and Content Hosting Technology

The solution hosts on INTEL or AMD processors using cloud-hosted server hardware on using various software formats for virtual machines. (e.g., Windows Server 2018.) Content, especially Video and other dynamic content is hosted by various media services (e.g. AZURE MEDIA SERVICE and BRIGHT COVE) All servers are set up in a high-availability fashion to ensure ultimate up-time, An active server takes in all the traffic and a warm backup server is on stand-by with continuous synchronization. This allows the solution to react to a server going down at any one point in time, the second server automatically kicks in without any manual intervention. Disaster recovery is also in place to ensure automatic failover in case of a data center outage.

### Solution Load Balancing for the Applications and Data

In this example of the solution, use of a 3^{rd} party (e.g., AZURE) hardware-based load balancing distributes end-user connections across a plurality of distributed servers. This enables greater resiliency, a balanced server load, with enhanced fault tolerance on the various applications deployed by this solution.

### Content Delivery Network

Content is held in a resilient blob storage that automatically replicates data to help guard against unexpected hardware failures. Content storage is triple-redundant with an option of geo-constrained redundant storage by jurisdictions that limit transfer of privacy related data. Content used in this context refers to content of all types and data of all types related to ABA treatment (e.g., educational literature, patient data and records, clinician data and records)

### Data Back-up and Security

The solution uses multiple levels of backups to ensure data resiliencies. Full backups of data are created daily with transactional hourly backups. Copies of the backups are also physically or virtually sent to off-site facilities to ensure the highest level of protection and resilience for customer data.

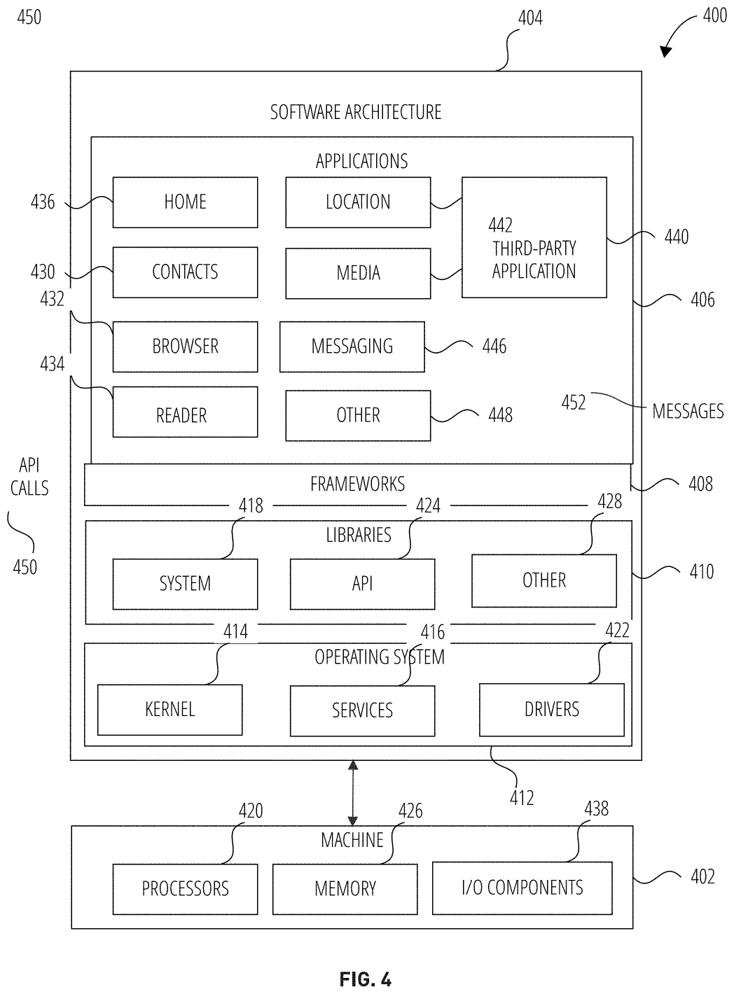

FIG. 4 is a solution block diagram 400 illustrating a software architecture 404 representative of the current solution, which can be installed on any one or more of the devices described herein. The software architecture 404 is supported by hardware such as a machine 402 that includes processors 420, memory 426, and I/O components 438. In this example, the software architecture 404 can be conceptualized as a stack of layers, where each layer provides a particular functionality. The software architecture 404 includes layers such as an operating system 412, libraries 410, frameworks 408, and applications 406. Operationally, the applications 406 invoke API calls 450 through the software stack and receive messages 452 in response to the API calls 450.

The operating system 412 manages hardware resources and provides common services. The operating system 412 includes, for example, a kernel 414, services 416, and drivers 422. The kernel 414 acts as an abstraction layer between the hardware and the other software layers. For example, the kernel 414 provides memory management, Processor management (e.g., scheduling), component management, networking, and security settings, among other functionality. The services 416 can provide other common services for the other software layers. The drivers 422 are responsible for controlling or interfacing with the underlying hardware. For instance, the drivers 422 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, power management drivers, and so forth.

The libraries 410 provide a low-level common infrastructure used by the applications 406. The libraries 410 can include system libraries 418 (e.g., C standard library) that provide functions such as memory allocation functions, string manipulation functions, mathematic functions, and the like. In addition, the libraries 410 can include API libraries 424 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 410 can also include a wide variety of other libraries 428 to provide many other APIs to the applications 406.

The frameworks 408 provide a high-level common infrastructure that is used by the applications 406. For example, the frameworks 408 provide various graphical user interface (GUI) functions, high-level resource management, and high-level location services. The frameworks 408 can provide a broad spectrum of other APIs that can be used by the applications 406, some of which may be specific to a particular operating system or platform.

In an example of the solution, the applications 406 comprise a home application 436, a contacts application 430, a browser application 432, a reader application 434 for recommended treatment or educational content, a location application 442 to capture where treatment is occurring, a media application 444, a messaging application 446, other application 448 where a facet of treatment is captured, and a broad assortment of other applications such as a third-party application 440. The applications 406 are programs that execute functions defined in the programs. Various programming languages can be employed to create one or more of the applications 406, structured in a variety of manners, such as object-oriented programming languages (e.g., Objective-C, Java, or C++) or procedural programming languages (e.g., C or assembly language). In a specific example, the third-party application 440 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 440 can invoke the API calls 450 provided by the operating system 412 to facilitate functionality described herein.

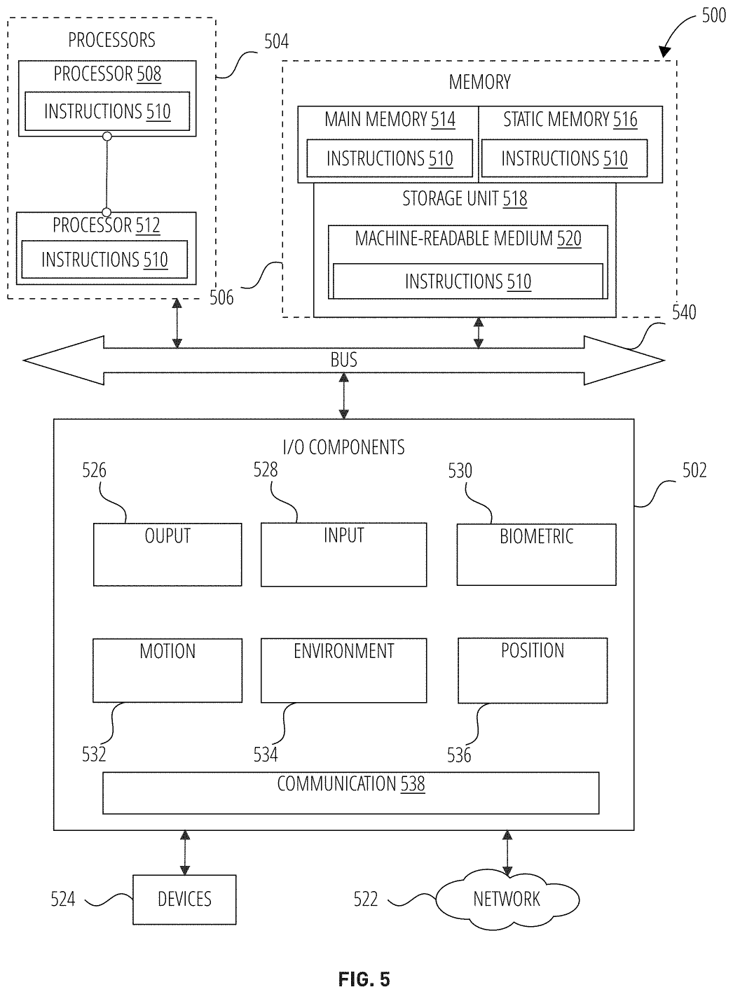

FIG. 5 is a diagrammatic representation of the solution machine 500 implementing the current solution within which instructions 510 (e.g., software, a program, an application, an applet, an app, or other executable code) for causing the machine 500 and its processors 508, 512 to perform any one or more of the methodologies discussed herein may be executed. For example, the instructions 510 may cause the machine 500 to execute any one or more of the methods described herein. The instructions 510 transform the general, non-programmed machine 500 into a particular machine 500 programmed to carry out the described and illustrated functions in the manner described. The machine 500 may operate as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a PDA, a cellular telephone, a smart phone, a mobile device, a wearable device, other smart devices, a web appliance, a network router, a network switch, a network bridge, or any machine capable of executing the instructions 510, sequentially or otherwise, that specify actions to be taken by the machine 500. Further, while only a single machine 500 is illustrated, the term “machine” shall also be taken to include a collection of machines that individually or jointly execute the instructions 510 to perform any one or more of the methodologies of this solution as discussed herein.

The machine 500 may include processors 504, memory 506, and I/O components 502, which may be configured to communicate with each other via a bus 540. In an example of the solution, the processors 504 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an ASIC, a Radio-Frequency Integrated Circuit (RFIC), another Processor, or any suitable combination thereof) may include, for example, a Processor 508 and a Processor 512 that execute the instructions 510. The term “Processor” is intended to include multi-core processors that may comprise two or more independent processors (sometimes referred to as “cores”) that may execute instructions contemporaneously. Although FIG. 5 shows multiple processors 504, the machine 500 may include a single Processor with a single core, a single Processor with multiple cores (e.g., a multi-core Processor), multiple processors with a single core, multiple processors with multiples cores, or any combination thereof.

The memory 506 includes a main memory 514, a static memory 516, and a storage unit 518, both accessible to the processors 504 via the bus 540. The main memory 514, the static memory 516, and storage unit 518 store the instructions 510 embodying any one or more of the methodologies or functions described herein. The instructions 510 may also reside, completely or partially, within the main memory 514, within the static memory 516, within machine-readable medium 520 within the storage unit 518 within at least one of the processors 504 (e.g., within the Processor's cache memory), or any suitable combination thereof, during execution thereof by the machine 500.

The I/O components 502 may include a wide variety of components to receive input, provide output, produce output, transmit information, exchange information, capture measurements, and so on. The specific I/O components 502 that are included in a particular machine will depend on the type of machine. For example, portable machines such as mobile phones may include a touch input device or other such input mechanisms, while a headless server machine will likely not include such a touch input device. It will be appreciated that the I/O components 502 may include many other components that are not shown in FIG. 5. In various example of the solutions, the I/O components 502 may include output components 526 and input components 528. The output components 526 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 528 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example of the solutions, the I/O components 502 may include biometric components 530, motion components 532, environmental components 534, or position components 536, among a wide array of other components. For example, the advanced biometric components 530 of this solution include components to detect expressions related to autism (e.g., hand expressions, facial expressions, vocal expressions, body gestures, or eye-tracking), measure bio-signals indicative of autism (e.g., blood pressure, heart rate, body temperature, perspiration, or brain waves), identify a personal individual characteristics related to autism (e.g., voice identification, retinal identification, facial identification, fingerprint identification, or electroencephalogram-based identification), and the like.

Communication may be implemented using a wide variety of technologies. The I/O components 502 further include communication components 538 operable to couple the machine 500 to a network 522 or devices 524 via respective coupling or connections. For example, the communication components 538 may include a network interface Component or another suitable device to interface with the network 522. In further examples, the communication components 538 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 524 may be another machine or any of a wide variety of peripheral devices (e.g., a peripheral device coupled via a USB).

Moreover, the communication components 538 may detect identifiers or include components operable to detect identifiers. For example, the communication components 538 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 538, such as location via Internet Protocol (IP) geolocation, location via Wi-Fi® signal triangulation, location via detecting an NFC beacon signal that may indicate a particular location, and so forth.

The various memories (e.g., main memory 514, static memory 516, and/or memory of the processors 504) and/or storage unit 518 may store one or more sets of instructions and data structures (e.g., software) embodying or used by any one or more of the methodologies or functions described herein. These instructions (e.g., the instructions 510), when executed by processors 504, cause various operations to implement the disclosed examples of the solutions.

The instructions 510 may be transmitted or received over the network 522, using a transmission medium, via a network interface device (e.g., a network interface component included in the communication components 536) and using any one of several well-known transfer protocols (e.g., hypertext transfer protocol (HTTP)). Similarly, the instructions 510 may be transmitted or received using a transmission medium via a coupling (e.g., a peer-to-peer coupling) to the devices 524.

As previously mentioned, this solution utilizes machine learning methodology to create a training solution for the data processed by each user and each client processed using this solution. By continuously measuring results of assessment outcomes and comparing it to the scoring algorithm, the present solution improves the predicted Assessment Output 116.

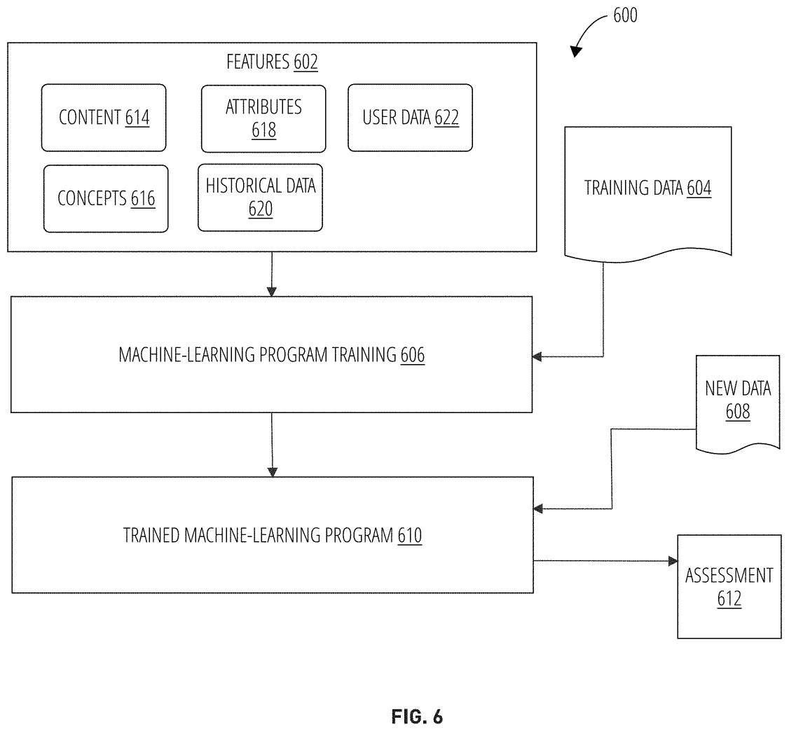

FIG. 6 illustrates training and use of a machine-learning program 600 by the present solution according to some example of the solutions. In some example of the solutions, machine-learning programs (MLPs), also referred to as machine-learning algorithms or tools, are used to perform operations associated with searches, such as job searches.

### Use of Machine Learning Algorithms to Improve ABA Results

By training the solution on a number of collected data sets, the solution improves its recommendations for patients that fall in to defined cohorts as described earlier. An example of the solution implementing a variety of MLPs is described below. Training of the solution selects combinations of training data comprising the following elements: historical data of ABA treatments, treatment variables, demographics of the patients, including cohort data, demographic of the patients parents, the demographics of the prescribers, the treatments the success of various ABA treatments, the frequency and limitations on which ABA treatments are paid for by insurance, data quality parameters reflecting the confidence and completeness of the data gathered, among other items listed in previous lists. The training of the solution improves the recommendations or inherent knowledge to be presented for the prescriber that are tailored to the patient's particular conditions. In another example of the solution, the MLPs are used to capture and train the solution in real time interaction with the patient, the patient's parents or care takers, and the prescribing clinician and any support staff. Depending on the MLPs incorporated on the mobile devices that may be used for data capture, a parent of the patient may use the functions of the mobile device MLP programs to capture audible, visual or other physical evidence that would be added to the ABA record related to the patient. Suitable privacy and de-identification filters would be applied to any data from any particular client or session to allow ongoing contribution of training data to the training corpus of historical data.

Machine learning is a field of study that gives computers the ability to learn without being explicitly programmed. Machine learning explores the study and construction of algorithms, also referred to herein as tools that may learn from existing data and make predictions about new data. Such machine-learning tools operate by building a model from example training data 604 in order to make data-driven predictions or decisions expressed as outputs or assessments (e.g., assessment 612). Although example of the solutions are presented with respect to a few machine-learning tools, the principles presented herein may be applied to other machine-learning tools.

In some example of the solutions, different machine-learning tools may be used. For example, Logistic Regression (LR), Naive-Bayes, Random Forest (RF), neural networks (NN), matrix factorization, and Support Vector Machines (SVM) tools may be used for classifying or scoring clients and their cohorts.

Two common types of problems in machine learning are classification problems and regression problems. Classification problems, also referred to as categorization problems, aim at classifying items into one of several category values (for example, is this object an apple or an orange?). Regression algorithms aim at quantifying some items (for example, by providing a value that is a real number).

The machine-learning algorithms use features 602 for analyzing the data to generate an assessment 612. Each of the features 602 is an individual measurable property of a phenomenon being observed. The concept of a feature is related to that of an explanatory variable used in statistical techniques such as linear regression. Choosing informative, discriminating, and independent features is important for the effective operation of the MLP in pattern recognition, classification, and regression. Features may be of different types, such as numeric features, strings, and graphs.

In one example of the solution, the features 602 may be of different types and may include one or more of content 614, concepts 616, attributes 618, historical data 622 and/or user data 620, merely for example.

The machine-learning algorithms use the training data 604 to find correlations among the identified features 602 that affect the outcome or assessment 612. In some example of the solutions, the training data 604 includes labeled data, which is known data for one or more identified features 602 and one or more outcomes, such as detecting communication patterns, detecting the meaning of the message, generating a summary of a message, detecting action items in messages detecting urgency in the message, detecting a relationship of the user to the sender, calculating score attributes, calculating message scores, etc.

With the training data 604 and the identified features 602, the machine-learning tool is trained at machine-learning program training 608. The machine-learning tool appraises the value of the features 602 as they correlate to the training data 604. The result of the training is the trained machine-learning program 610.

When the trained machine-learning program 610 is used to perform an assessment, new data 606 is provided as an input to the trained machine-learning program 610, and the trained machine-learning program 610 generates the assessment 612 as output.

### Interface Examples

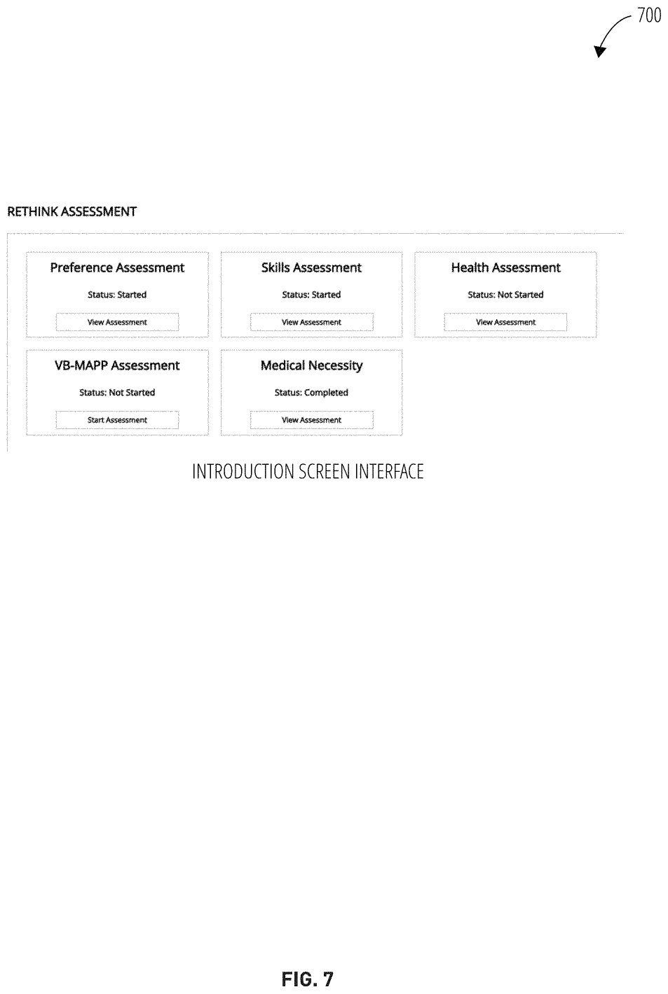

The Introduction Screen User Interface 700 is presented in FIG. 7 showing basic choices as presented in the Assessment Flow 100 FIG. 1. This allows the user to select the type of assessment to be performed.

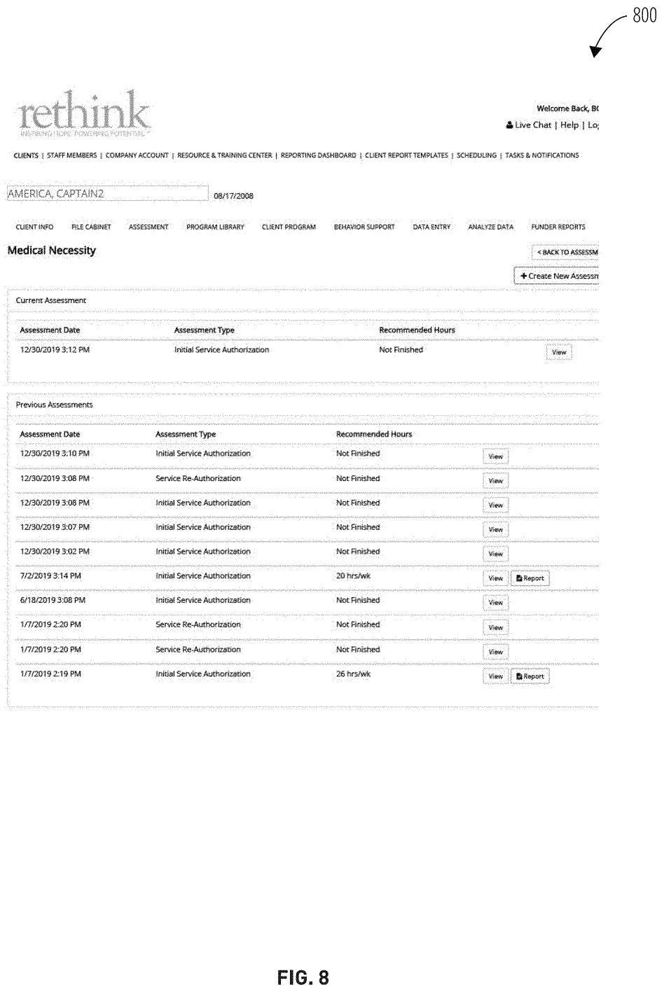

Open Assessment User Interface 800 as shown in FIG. 8 tracks all of the open assessments being made by the solution user. This interface example allows the user to quickly browse as to which assessment has a work requirement.

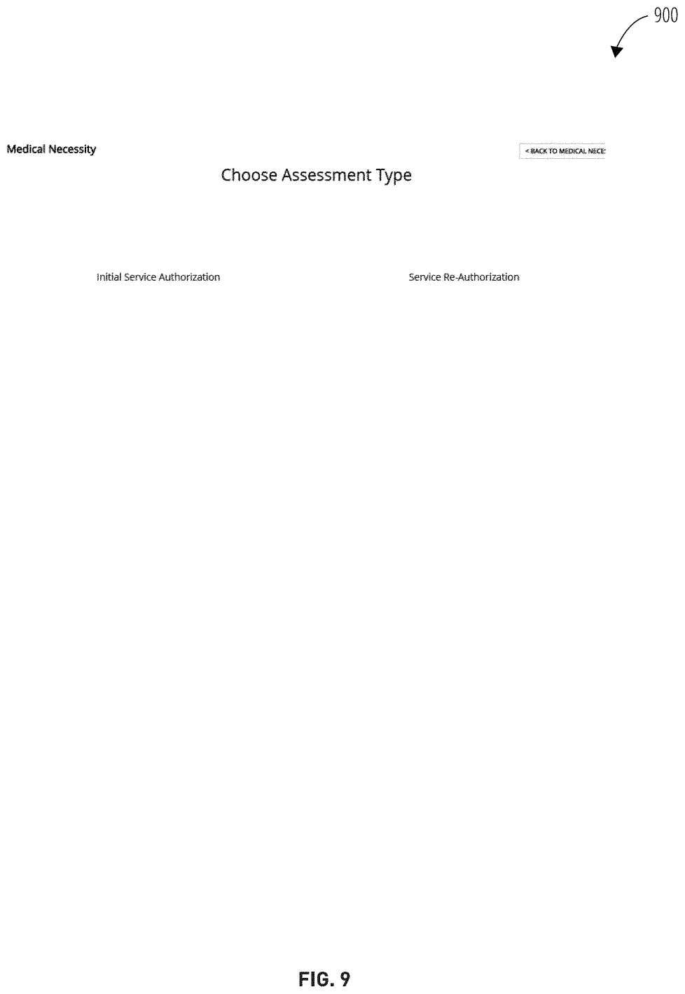

Different assessment choices are available in Assessment Choice User Interface 900 shown in FIG. 9. Assessment authorization varies dependent upon a service being initialized or a service being reauthorized.

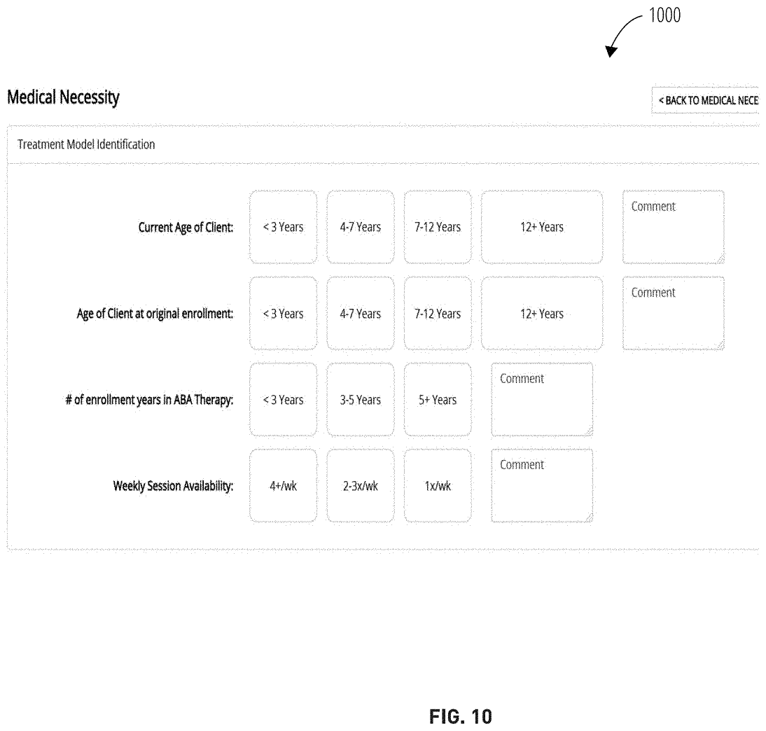

FIG. 10 identifies the variable models in Treatment Model Input User Interface 1000 showing dimensions of Age, length of treatment, Age of Client at enrollment, and the periodicity of treatments available.

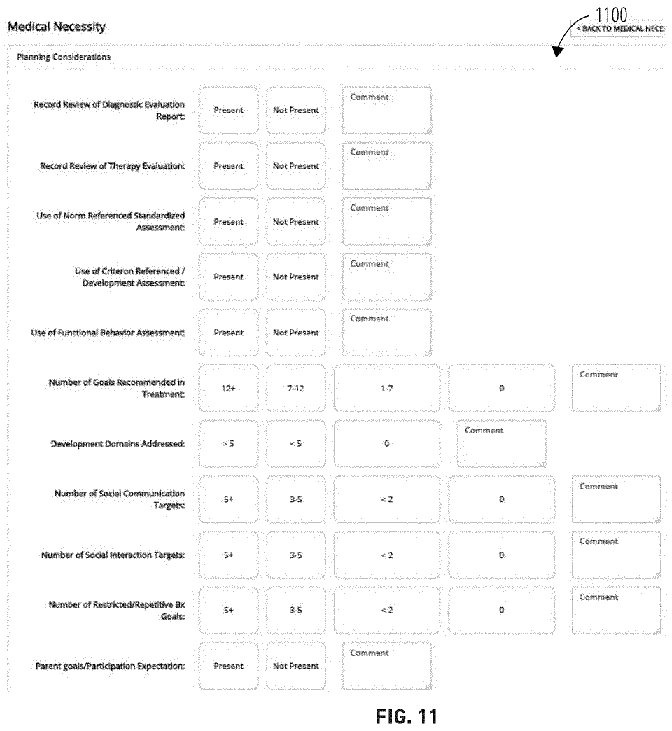

FIG. 11 shows many of the planning considerations inherent in an ABA model by Planning Data User Interface 1100. This interface exemplifies the complexity of managing and recording ABA treatments.

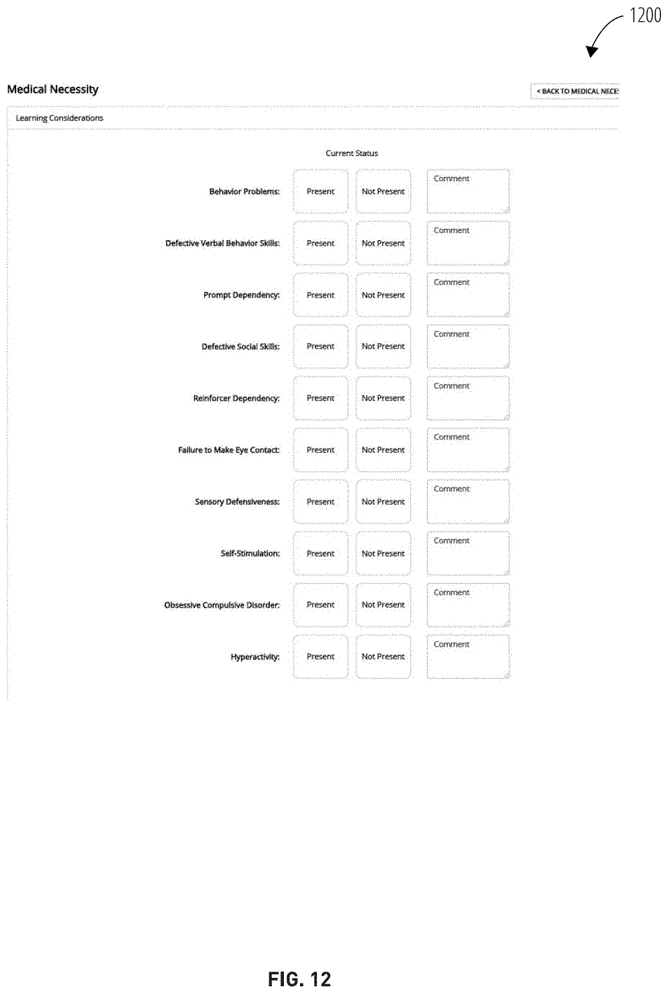

FIG. 12 demonstrates the multifactor learning considerations in executing the Current Status User Interface 1200. This interface also captures the various biometric components 530 that may be used to augment a professional psychologist evaluation for an ABA regimen.

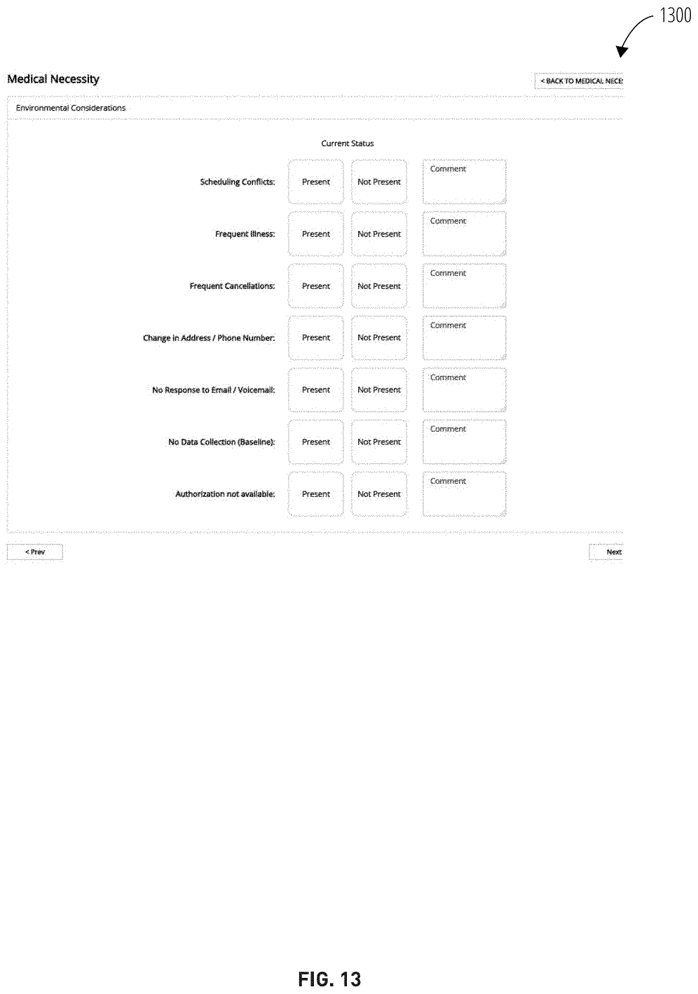

FIG. 13 shows the Environmental Considerations User Interface 1300 captures the current environmental conditions for the subject considered for the ABA model to be engaged on the subject.

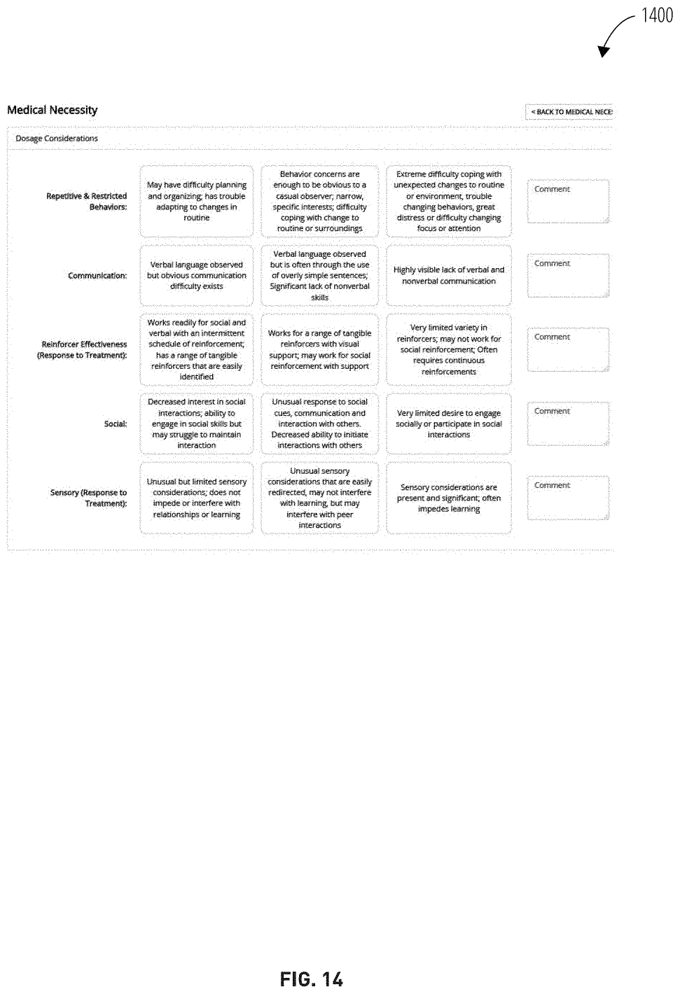

Clinical Data User Interface 1400 establishes the dosage baselines and modifications to the baseline in FIG. 14.

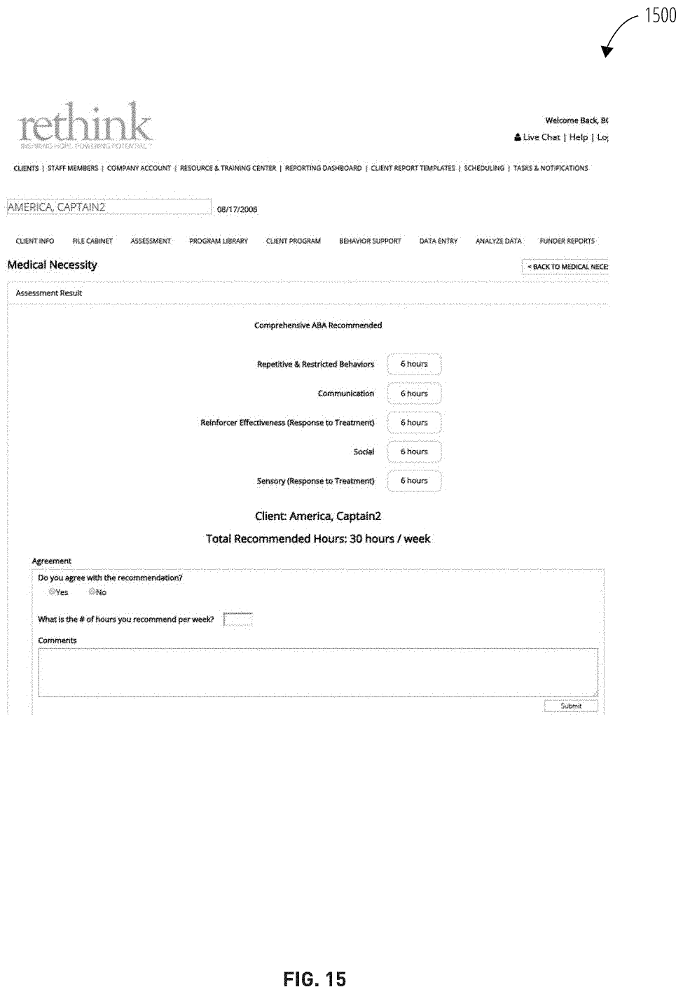

FIG. 15 is a Result and Recommendation User Interface 1500 example of the solution. This a view of a Comprehensive ABA recommendation prescribing treatment in hours across five categories. This interface also captures agreement of the clinician with the recommendation. This is the first step in the feedback of improving the recommendation result.

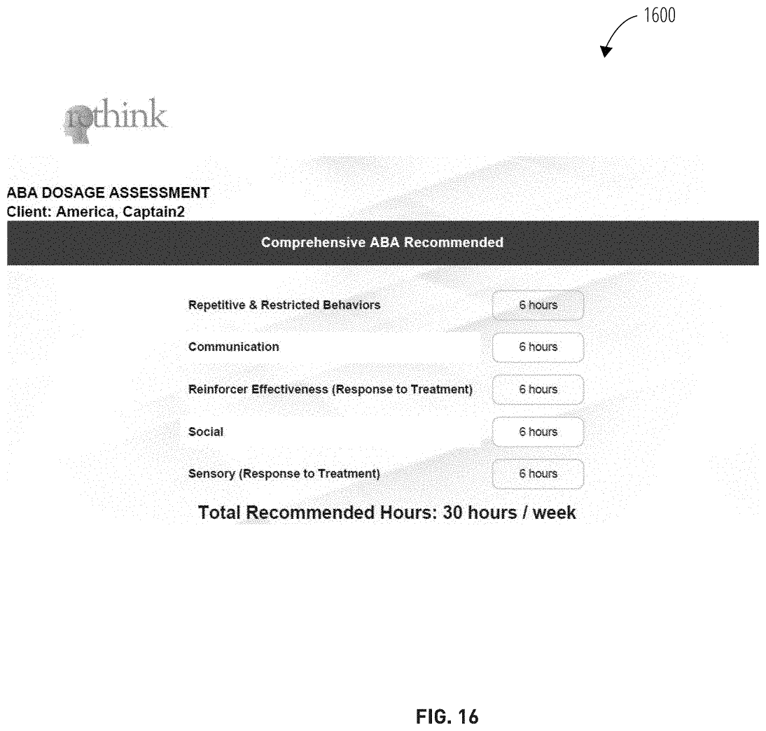

FIG. 16 shows Results Summary User Interface 1600 shows an example of the solution showing a separate example of a result summary that would be shown to a payer or guardian of the patient in a Comprehensive ABA recommendation.

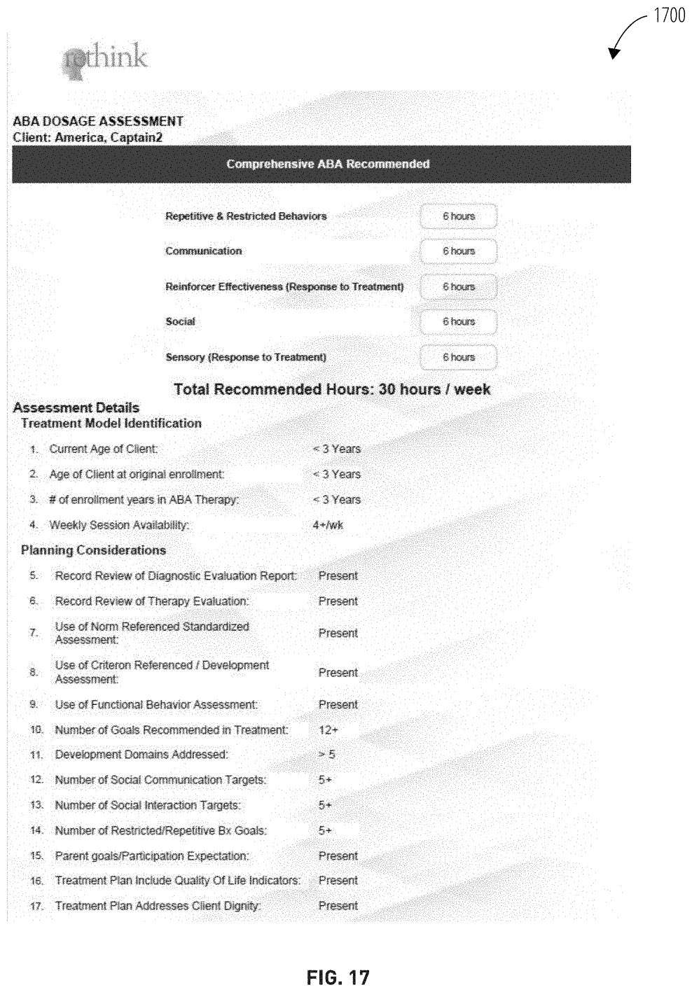

Comprehensive Report User Interface 1700 example is partially shown in FIG. 17. This interface summarizes all of the unique considerations captured by the Current Status User Interface 1200 solution for the individual in concordance with the views of the practitioner in a manner most appropriate for the Payer.

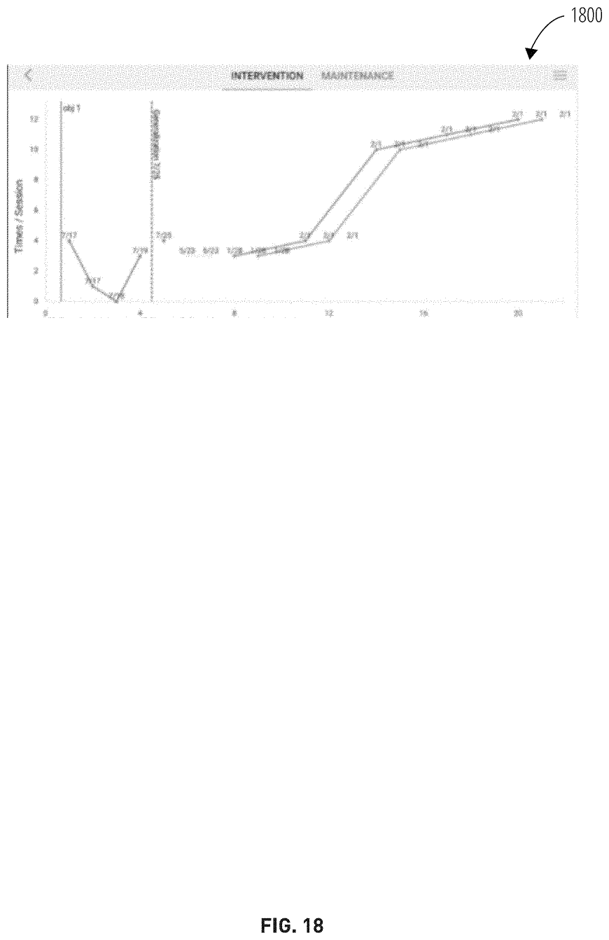

A charting interface example 1800 is shown in FIG. 18 showing the progression of intervention and the charting of ABA therapy targets and the execution of the actual therapy. This interface view can be used to represent various expected ABA therapy goals and inputs/transformations/outputs.

The evidence supporting the use of behavior analysis in children with autism up to the age of twelve is compelling. While limitations in research are present regarding the effects of behavior analysis as it pertains to population health management, the overwhelming support for covering these services remains. Additionally, in the recent years there has been a strong effort to remove funding-based treatment decisions done on behalf of managed care organizations and increase access to proper dosage recommendation by the clinician influenced best practice standards.

The challenge in utilization review may simply be a complex language barrier between the treatment providers and managed care organizations For example, the behavior analyst has a priority to their individual clients whereas the managed health care organizations have a priority to whole patient populations. A collaborative effort to understand one another's fiscal, clinical and patient outcome needs will continue to be of great importance. This also presents us with a unique opportunity to create clinical job aids pertaining to treatment intensity recommendations that may also serve to promote standards of care amongst treating providers nationwide and drive the continued research on the individual features of ABA treatment for individuals with autism of all ages.

The above description includes references to the accompanying drawings, which form a part of the detailed description. The drawings show, by way of illustration, specific examples of the solutions in which the invention can be practiced. These variants of the solutions are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a Computer-Readable Medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other variants of the solutions can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed variant of the solution. Thus, the following claims are hereby incorporated into the Detailed Description as examples or variants of the solutions, with each claim standing on its own as a separate variant of the solution, and it is contemplated that such variant of the solutions can be combined with each other in various combinations or permutations. The scope of the invention should be determined with reference to the appended claims, along with the full scope of equivalents to which such claims are entitled.

What is claimed is:

1. A computer-implemented behavioral health assessment for ABA therapy method comprising:
receiving a set of responses from a queried condition of an individual to generate an ABA score established by a human-generated examination of the individual;
prescribing an ABA treatment based on the ABA score;
correlating, via a processor, the ABA treatment to a corpus of ABA treatment data; and
calibrating the recommended ABA treatment based on a set of computer-implemented feedback associated with the ABA score associated with the individual, via a processor, providing at least an ABA treatment recommendation for modification of the ABA treatment.

2. The computer-implemented behavioral health assessment for ABA therapy method of claim 1 further comprising receiving new data on the progress of the ABA treatment associated with the individual under treatment, during the ABA treatment period and updating a profile associated with the individual receiving the ABA treatment.

3. The computer-implemented behavioral health assessment for ABA therapy method of claim 1 further comprising updating the corpus of ABA treatment data with a set of de-identified data of the individual during the ABA treatment.

4. The computer-implemented behavioral health assessment for ABA therapy method of claim 3 further comprising updating the ABA treatment recommendation to enhance the ABA treatment in process.

5. The computer-implemented behavioral health assessment for ABA therapy method of claim 4 further comprising alerting changes in an outcome of the ABA treatment.

6. The computer-implemented behavioral health assessment for ABA therapy method of claim 4 further comprising the updated ABA treatment recommendation wherein a content suggestion for the individual receiving the ABA treatment is included.

7. The computer-implemented behavioral health assessment for ABA therapy method of claim 4 further comprising the updated ABA treatment recommendation wherein a time extension suggestion is recommended for the individual receiving the ABA treatment.

8. The computer-implemented behavioral health assessment for ABA therapy method of claim 4 further comprising the updated ABA treatment recommendation wherein an activity modification suggestion is recommended for the individual receiving the ABA treatment.

9. The computer-implemented behavioral health assessment for ABA therapy method of claim 4 further comprising the updated ABA treatment recommendation wherein a new activity suggestion is recommended for the individual receiving the ABA treatment.

10. The computer-implemented behavioral health assessment for ABA therapy method of claim 4 further comprising the updated ABA treatment recommendation wherein an educational component suggestion is recommended for the individual receiving the ABA treatment.

11. The computer-implemented behavioral health assessment for ABA therapy method of claim 4 further comprising the updated ABA treatment recommendation wherein a medical necessity score update suggestion is recommended for the parents of the individual receiving the ABA treatment.

12. The computer-implemented behavioral health assessment for ABA therapy method of claim 4 further comprising the updated ABA treatment recommendation wherein an educational component suggestion is recommended for the parents of the individual receiving the ABA treatment.

13. One or more non-transitory computer readable media comprising instructions that, when executed with a computer system configured to execute instructions with binary data, cause the computer system to at least:
receive, by a computer system, a set of binary data correlated to an individual behavioral condition captured in a questionnaire;
correlate, by the computer system, that the set of binary data to a score representative of an ABA recommended treatment;
execute, by the computer system, the score further comprising a set of ABA treatment plans based on the score;
recalibrate, by the computer system, the correlation between the set of binary data and the score that recommends the set of ABA treatment plans; and
recommend, by the computer system, a recommendation for the ABA recommended treatment based on the score and the binary set of data.

14. The one or more non-transitory computer readable media of claim 13 further comprising: receipt of a set of new data on the progress of the ABA treatment associated with an individual under treatment, during the ABA treatment period and updating a treatment profile associated with the individual receiving the ABA treatment.

15. The one or more non-transitory computer readable media of claim 13 further comprises an addition of the recommendation to a corpus of ABA recommended treatment data with the set of binary data of the individual behavioral condition under the ABA treatment.

16. The one or more non-transitory computer readable media of claim 15 further comprises a change to the recommendation of the ABA recommended treatment to enhance the ABA recommended treatment.

17. The one or more non-transitory computer readable media of claim 16 further comprises a creation of a treatment alert based on a change in a measured outcome of the ABA recommended treatment.

18. The one or more non-transitory computer readable media of claim 16 further comprises the change to the ABA recommended treatment wherein a content suggestion is made as part of the ABA recommended treatment.

19. The one or more non-transitory computer readable media of claim 16 further comprises the updated ABA treatment recommendation wherein a time extension suggestion is recommended for the individual receiving the ABA recommended treatment.

20. The one or more non-transitory computer readable media of claim 16 further comprises the updated ABA treatment recommendation wherein an activity modification suggestion is recommended for the individual treated by the ABA recommended treatment.
